(19) United States
(12) Patent Application Publication
(10) Pub. No.: US 2021/0315977 A1
(43) Pub. Date: Oct. 14, 2021

(54) THERMOSTABLE FORMULATION OF A21G HUMAN INSULIN

(71) Applicant: ADOCIA, Lyon (FR)

(72) Inventors: You-Ping CHAN, Ternay (FR); Fany DI LORENZO, Lyon (FR)

(73) Assignee: ADOCIA, Lyon (FR)

(21) Appl. No.: 17/259,948

(22) PCT Filed: Jul. 15, 2019
(86) PCT No.: PCT/EP2019/069049

§ 371 Date: Feb. 11, 2021

(30) Foreign Application Priority Data
Jul. 13, 2018 (FR) 18/56479

(51) Int. Cl. A61K 38/28; A61K 9/08; A61K 9/00; A61K 47/10; A61K 47/26; A61K 47/18

(52) U.S. Cl. CPC A61K 38/28; A61K 9/08; A61K 9/0019; A61K 47/18; A61K 47/26; A61K 47/183; A61K 47/10

(57) ABSTRACT

A composition in the form of an injectable aqueous solution, the pH of which is between 7.2 and 8.0 (7.2<pH<8.0) and which includes at least A21G human insulin, the composition being intended to be used in a method for treating diabetes, wherein it is administered as a bolus before meals.

The present invention relates to a composition in the form of an injectable aqueous solution, the pH of which is between 7.2 and 8.0, comprising at least A21G human insulin. Said composition has improved physical and/or chemical stability, in particular at 40 and 50° C. Human insulin and its analogs, produced by genetic engineering since the early 1980s, have revolutionized the treatment of diabetic patients in need of insulin-based therapy.

Compliance and treatment can be further improved by the choice of insulins, rapid or basal, as well as by the choice of injection devices, such as pre-filled pens or pumps.

In most developed countries, access to insulin is easy and a person with diabetes can, in general, lead a “normal” life.

All currently marketed insulins must be stored at a temperature of between 2 and 8° C. After the first use, most insulins can be stored for about 1 month at a maximum temperature of 30° C. (Grajower et al. Diabetes Care 2003, 26, 2665-2669).

When using an injection device of the pump type, the reservoir comprising the insulin may be in contact with a surface the temperature of which is greater than 30° C. This is particularly the case when using an implantable pump, the reservoir of which is placed under the skin. One of the problems which the present invention aims to solve may be the development of a thermally stable insulin formulation, in particular at temperatures above 30° C., and in particular around 37° C.

In addition, in certain regions of the world, the average temperature is of the order of 25° C. and can very regularly exceed 30° C. and even locally reach 40° C. to 45° C. during the day. The use of earthenware pots, common in some hot countries, makes it possible to reduce the average temperature by 3 to 8° C. but this is often insufficient to store an insulin-based composition for more than a month of (Ogle et al. al. Diabet. Med. 2016, 33, 1544-1553).

Thus, in certain cases, insulin is difficult to access for part of the population, because of its price and the problems of maintaining the cold chain. This is particularly glaring in the case of regions with a low standard of living, having a hot or even tropical climate, and/or far from centers benefiting from infrastructures allowing good preservation of insulin.

Therefore, one of the problems to be solved is to improve access to insulin to a higher number of patients thanks to an insulin formulation having improved stability at high temperature, in particular compared to insulins currently marketed. It is also advantageous if this formulation can be inexpensive. A thermally stable insulin formulation over a relatively long period of time can also be useful in areas without electricity, in war zones or in case of a natural disaster.

It is known that the chemical and physical stability of insulins can be partly dependent on their primary structure or sequence, on the pH and on the nature of the excipients.

As examples chosen from commercial products, the following products may be mentioned.

Human insulin, as marketed in a vial and formulated at pH 7.4, does not make it possible to obtain a formulation that is stable at 40° C. for more than 3 weeks because it is physically unstable. Thus, beyond this period, the specifications of the European Pharmacopoeia for insulin are no longer respected. In particular, the level of chemical aggregates quickly exceeds 2%, and the recovery is less than 90%. At 50° C., human insulin is not stable beyond a few days based on chemical stability. Physical stability at 50° C. is less than two weeks. The manufacturer recommends storing this product for a maximum of 31 days at a temperature not exceeding 30° C.

Insulin lispro, an analog of human insulin and formulated at pH 7.4, which has a lysine residue at position B28 and a proline residue at position B29, is a prandial insulin marketed under the name Humalog®, and is physically stable for less than a week at 50° C. The manufacturer recommends storing this product for a maximum of 28 days at a temperature not exceeding 30° C.

Insulin glulisine, an analog of human insulin and formulated at pH 7.3, which has a lysine residue at position B3 and a glutamic acid residue at position B29, is a prandial insulin marketed under the name Apidra®. The manufacturer recommends storing this product for a maximum of 28 days at a temperature not exceeding 25° C.

Insulin glargine, an analog of human insulin and formulated at pH 4.0, which has a glycine residue at position A21 and two arginine residues at position B30 and B31, is a basal insulin marketed under the name Lantus®, and is physically stable for less than a week at 50° C. The manufacturer recommends storing this product for a maximum of 28 days at a temperature not exceeding 30° C.

Numerous studies have focused on the role of excipients in improving the stability of compositions comprising insulin. More specifically they describe that zinc and preservatives can play an important role in this regard. For a review on the stability of insulin, it is possible to refer to the following publications by Brange J. et al.:
Insulin Structure and Stability. In: Wang Y. J., Pearlman R. (eds) Stability and Characterization of Protein and Peptide Drugs. Pharmaceutical Biotechnology, Vol 5. Springer, Boston, Mass.) 1993,
Chemical Stability of Insulin. 1. Pharmaceutical Research 1992, 9, 715-726,
Chemical Stability of Insulin. 2. Pharmaceutical Research 1992, 9, 727-734,
Chemical Stability of Insulin. 3. Acta. Pharm. Nord. 1992, 4, 149-158,
Chemical Stability of Insulin. 4. Acta. Pharm. Nord. 1992, 4, 209-222.

Known routes for the chemical degradation of human insulin are, in particular, the deamidation of asparagines at B3 and A21, the formation of covalent aggregates, and the rupture and rearrangement of disulfide bridges. These degradation modes are dependent, at least in part, on the pH of the formulation and on the presence of excipients. Deamidation at A21 is predominant at acidic pH while deamidation at B3 is greater at neutral pH.

With regard to physical stability, it is known that insulins can form fibrils or aggregates, in particular by heating or by shaking. Since these fibrils or aggregates can be immunogenic, they must therefore be avoided.

Obtaining a physically and chemically stable insulin over a longer period of time at temperatures above 30° C., in particular at 40 and/or at 50° C., is a challenge because the chemical degradation and the formation of fibrils or aggregates are caused by distinct phenomena whose mechanisms, kinetics and triggering factors are not all known. Indeed, even if the main impurities resulting from the degradation of insulins are known, there remain many impurities that are unidentified and consequently, resulting from unknown mechanism(s).

Understanding some of the degradation modes makes it possible to provide certain solutions for improving physical and/or chemical stability. However, significant improvements having a practical interest, in particular leading to a physical stability greater than one month at 40° C. and allowing at least postprandial insulin therapy, have not been disclosed to date.

In this context, a prandial insulin formulation having good physical and/or chemical stability at temperatures above 30° C., such as 40 and 50° C., would be very interesting. In particular, a “thermostable” prandial insulin formulation having the following characteristics would be a very advantageous product compared to all the products currently marketed:
physical and chemical stability for at least one month at 40° C. and at least 2 weeks at 50° C.,
good compatibility with anti-microbial preservatives,
a possible use in vials (bottles) and/or cartridges,
pharmacokinetic profile similar to that of human insulin at the same concentration, and
ease of manufacture.

Surprisingly, the applicant has developed a composition, in the form of an injectable aqueous solution, comprising an A21G human insulin at a pH between 7.2 and 8.0 having a physical and/or chemical stability at 40° C. for at least 3 weeks and/or at 50° C. for at least one week.

It should be noted that the precision of the measurement of the pH value is ±0.1.

According to one embodiment, the pH of the compositions is between 7.2 and 8.0.

According to another embodiment, the pH of the compositions is between 7.2 and 7.8.

According to yet another embodiment, the pH of the compositions is between 7.2 and 7.6.

According to one embodiment, the composition is physically and/or chemically stable for at least one month (i.e., 4 weeks) at 40° C. and at least two weeks at 50° C.

According to one embodiment, the composition is physically stable for at least one month at 40° C. and for at least two weeks at 50° C.

According to one embodiment, the composition is chemically stable for at least one month (i.e., 4 weeks) at 40° C. and at least two weeks at 50° C.

According to one embodiment, the composition is chemically stable for at least 4 weeks at 40° C.

According to one embodiment, the composition is chemically stable for at least 8 weeks at 40° C.

According to one embodiment, the composition is chemically stable for at least 12 weeks at 40° C.

According to one embodiment, the composition is chemically stable for at least two weeks at 50° C.

According to one embodiment, the composition is physically stable for at least 8 weeks at 40° C.

According to one embodiment, the composition is physically stable for at least 12 weeks at 40° C.

According to one embodiment, the composition is physically stable for at least 16 weeks at 40° C.

According to one embodiment, the composition is physically stable for at least 4 weeks at 50° C.

According to one embodiment, the composition is physically stable for at least 6 weeks at 50° C.

According to one embodiment, the composition is physically stable for at least 8 weeks at 50° C.

According to one embodiment, the composition is physically stable for at least 12 weeks at 50° C.

The term “thermostable formulation” is understood to mean a formulation having a physical and chemical stability of at least 4 weeks at 40° C. and of at least 2 weeks at 50° C.

The term “chemically stable composition” is understood to mean a composition in which the insulin recovery is of at least 90% and in which the HMWP level (“aggregates”) is less than 2%.

The term “physically stable composition” is understood to mean compositions which meet the criteria of visual inspection described in the European, American and international pharmacopoeia, i.e., compositions which are clear and which do not contain visible particles, but are also colorless.

The term “injectable aqueous solution” is understood to mean water-based solutions which meet the conditions of the EP and US pharmacopoeias.

The compositions in the form of an injectable aqueous solution according to the invention are clear solutions. The term “clear solution” is understood to mean compositions which meet the criteria described in the American and European pharmacopoeias relating to injectable solutions. In the US Pharmacopoeia, these solutions are defined in part <1151> referring to injection (<1>) (referring to <788> according to USP 35 and specified in <788> according to USP 35 and in <787>, <788> and <790> USP 38 (as of 1 Aug. 2014), per USP 38). In the European Pharmacopoeia, injectable solutions must meet the criteria laid down in Sections 2.9.19 and 2.9.20.

The term “monthly” or “one month” is understood to mean a period of 4 weeks.

The term “bimonthly” is understood to mean a period of 2 weeks.

In the examples, the term “dB3 impurity” is understood to mean an insulin in which the asparagine located at the third position of the B chain is deamidated and converted into aspartic acid.

In the examples, the term “disoB3 impurity” is understood to mean an insulin in which the asparagine located at the third position of the B chain is deamidated and converted into an isoaspartic acid.

A21G human insulin is an insulin which has the same sequence as human insulin but in which the asparagine at position A21 is mutated to glycine. In other words, this A21G human insulin has a sequence identical to that of human insulin, with the exception of position A21 where a glycine residue replaces the asparagine residue present in human insulin.

The sequence of chain A is SEQ ID NO: 1 GIVEQCCTSICSLYQLENYCG and the sequence of chain B is SEQ ID NO: 2 FVNQHLCGSHLVEALYLVCGERGFFYTPKT.

This A21G human insulin is the main metabolite of insulin glargine, marketed under the name Lantus® (SANOFI) (Bolli et al. Diabetes Care 2012, 35, 2626-2630). This insulin has a duration of action similar to that of human insulin and can thus be used during meals, it is therefore a so-called “prandial” insulin.

Obtaining a composition in the form of an injectable aqueous solution having improved physical and chemical stability properties compared to those of the prandial insulins disclosed in the prior art at temperatures above 30° C. and at a pH close to 7, is remarkable because it is well known to a person skilled in the art that the physical and chemical stability properties are very difficult to predict, especially in the case of combinations.

The invention also relates to containers comprising the composition according to the invention. These containers may be vials, cartridges, pre-filled syringes, and reservoirs. Said containers may be included or enclosed in injection pens or in insulin pumps.

The invention also relates to compositions for their use as a medicament.

The invention also relates to compositions for their use in the treatment of diabetes.

According to one embodiment, it is type I diabetes.

According to one embodiment, it is type II diabetes.

The invention also relates to pharmaceutical formulations comprising such compositions.

The invention also relates to the use of a composition according to the invention.

The invention also relates to a method for treating diabetes comprising the administration of a composition according to the invention. It relates more particularly to a method comprising the administration of a composition according to the invention as prandial insulin.

According to one embodiment, it is a treatment method intended to treat human beings.

The invention also relates to a composition according to the invention intended for use in a method for treating diabetes, characterized in that it is administered as a bolus before meals.

It relates more particularly to a composition intended for use in a method for treating diabetes, characterized in that it is administered as prandial insulin.

According to one embodiment, A21G human insulin is in hexameric form.

In the present application, 100 U of A21G human insulin correspond to 3.5 mg.

In one embodiment, the concentration of A21G human insulin is between 240 and 6000 μM or between 40 and 1000 U/mL.

In one embodiment, the concentration of A21G human insulin is between 240 and 3000 μM or between 40 and 500 U/mL.

In one embodiment, the concentration of A21G human insulin is 600 μM or 100 U/mL.

In one embodiment, the concentration of A21G human insulin is 1200 μM or 200 U/m L.

In one embodiment, the concentration of A21G human insulin is 1800 μM or 300 U/m L.

In one embodiment, the concentration of A21G human insulin is 2400 μM or 400 U/m L.

In one embodiment, the concentration of A21G human insulin is 3000 μM or 500 U/m L.

In one embodiment, the concentration of A21G human insulin is 3600 μM or 600 U/m L.

In one embodiment, the concentration of A21G human insulin is 4200 μM or 700 U/m L.

In one embodiment, the concentration of A21G human insulin is 4800 μM or 800 U/m L.

In one embodiment, the concentration of A21G human insulin is 5400 μM or 900 U/m L.

In one embodiment, the concentration of A21G human insulin is 6000 μM or 1000 U/mL.

In one embodiment, the compositions according to the invention further comprise zinc salts.

In one embodiment, the zinc comes from ZnCl₂ or ZnO.

In one embodiment, the compositions according to the invention comprise zinc salts at a concentration of between 50 and 600 μM per 100 U of A21G human insulin.

In one embodiment, the compositions according to the invention comprise zinc salts at a concentration of between 100 and 500 μM per 100 U of A21G human insulin.

In one embodiment, the compositions according to the invention comprise zinc salts at a concentration of between 150 and 400 μM per 100 U of A21G human insulin.

In one embodiment, the compositions according to the invention comprise zinc salts at a concentration of between 200 and 350 μM per 100 U of A21G human insulin.

In one embodiment, the compositions according to the invention comprise zinc salts at a concentration of between 200 and 300 μM per 100 U of A21G human insulin.

In one embodiment, the compositions according to the invention comprise zinc salts at a concentration of 230 μM per 100 U of A21G human insulin.

In one embodiment, the compositions according to the invention further comprise at least one buffer.

In one embodiment, the compositions according to the invention comprise a buffer chosen from the group consisting of a sodium acetate buffer and a Tris buffer, abbreviation of trishydroxymethylaminomethane.

In one embodiment, the compositions according to the invention comprise a Tris buffer.

In one embodiment, the trishydroxymethylaminomethane is present at a concentration of between 2 and 100 mM.

In one embodiment, the trishydroxymethylaminomethane is present at a concentration of between 4 and 80 mM.

In one embodiment, the trishydroxymethylaminomethane is present at a concentration of between 5 and 50 mM.

In one embodiment, the trishydroxymethylaminomethane is present at a concentration of between 6 and 40 mM.

In one embodiment, the trishydroxymethylaminomethane is present at a concentration of between 6 and 20 mM.

In one embodiment, the compositions according to the invention comprise arginine.

In one embodiment, the arginine is present at a concentration of between 5 and 100 mM.

In one embodiment, the arginine is present at a concentration of between 10 and 90 mM.

In one embodiment, the arginine is present at a concentration of between 20 and 80 mM.

In one embodiment, the arginine is present at a concentration of between 30 and 70 mM.

In one embodiment, the arginine is present at a concentration of between 40 and 60 mM.

In one embodiment, the compositions according to the invention comprise arginine and a Tris buffer.

In one embodiment, the compositions according to the invention further comprise preservatives.

In one embodiment, the preservatives are phenolic preservatives.

In one embodiment, the phenolic preservatives are chosen from the group consisting of m-cresol and phenol, alone or as a mixture.

In one embodiment, the concentration of phenolic preservative(s) is between 10 and 100 mM.

In one embodiment, the concentration of phenolic preservative(s) is between 15 and 100 mM.

In one embodiment, the concentration of phenolic preservative(s) is between 20 and 75 mM.

In one embodiment, the concentration of phenolic preservative(s) is between 20 and 60 mM.

In one embodiment, the preservative is phenol.

In one embodiment, the concentration of phenol is between 30 and 75 mM.

In one embodiment, the concentration of phenol is between 40 and 60 mM.

In one embodiment, the concentration of phenol is 50 mM.

In one embodiment, the preservative is m-cresol.

In one embodiment, the concentration of m-cresol is between 15 and 50 mM.

In one embodiment, the m-cresol concentration is between 20 and 30 mM.

In one embodiment, the concentration of m-cresol is 25 mM.

In one embodiment, the composition comprises a mixture of m-cresol and phenol.

In one embodiment, the total concentration of m-cresol and phenol is between 20 and 75 mM.

According to one embodiment, the composition is free of zinc chelating agent.

The term “zinc chelating agent” is understood to mean an agent which is capable of complexing zinc. This agent may have a metal binding stability constant log K for zinc of at least 4.5 determined at 25° C.

The metal binding stability constants listed in the “National Institute of Standards and Technology Reference Database 46 (Critically Selected Stability Constants of Metal Complexes) can be used. This database lists the log K constants determined at 25° C. Therefore, the compliance of a chelating agent within the meaning of the present invention can be determined on the basis of its binding stability constant for zinc, measured at 25° C., and as described in this database.

Mention may be made, among the zinc chelating agents, of polydentate organic anions.

According to one embodiment, the chelating agent is chosen from the following compounds: EDTA (log K=14.5), citrate, (log K=4.93), EGTA (log K=12.6), pyrophosphate (log K−8.71) and alginate (log K=6.91).

According to another embodiment, the chelating agent is a compound comprising a pair of free electrons or an electron density permitting an interaction with ionic zinc. Such compounds can be polydentate amines. These can be chosen from the following compounds: ethylene diamine, aromatic or heteroaromatic substances, in particular those comprising an imidazole unit, such as histidine (log K=6.91).

In one embodiment, the compositions according to the invention further comprise a surfactant.

In one embodiment, the surfactant is selected from the group consisting of Poloxamer 188, and polysorbates, in particular Tween® 20, also called Polysorbate 20, and Tween® 80, also called Polysorbate 80.

In one embodiment, the surfactant is chosen from polysorbates.

In one embodiment the polysorbate concentration is between 4 and 20 μM (4 μM<polysorbate concentration 20 μM).

In one embodiment, the surfactant is chosen from Tween® 20, also called Polysorbate 20, and Tween® 80, also called Polysorbate 80.

In one embodiment, the surfactant is Tween® 20, also called Polysorbate 20.

In one embodiment, the concentration of Tween® 20 is between 4 and 20 μM.

In one embodiment, the concentration of Tween® 20 is between 5 and 10 μM.

In one embodiment, the concentration of Tween® 20 is 8 μM.

In one embodiment, the Poloxamer 188 concentration is between 0.5 and 2.5 mg/mL.

In one embodiment, the Poloxamer 188 concentration is between 0.8 and 1.6 mg/mL.

In one embodiment, the Poloxamer 188 concentration is 1.2 mg/mL.

According to one embodiment, the composition is free of polysorbate 80.

According to one embodiment, the composition is free of at least one zinc chelating agent and polysorbate 80.

In one embodiment, the composition comprises Tween® 20 and a Tris buffer.

In one embodiment, the invention relates to a composition in the form of an injectable aqueous solution, the pH of which is from 7.2 to 7.6, comprising at least A21G human insulin, a surfactant and a buffer. According to one embodiment, it is Tween® 20 and a Tris buffer.

In one embodiment, the invention relates to a composition in the form of an injectable aqueous solution, the pH of which is 7.4, comprising at least A21G human insulin, a surfactant and a buffer. According to one embodiment, it is Tween® 20 and a Tris buffer.

The compositions according to the invention may also comprise additives such as tonicity agents, also called osmotic agents.

In one embodiment, the tonicity agents are selected from the group consisting of glycerin, sodium chloride, mannitol, trehalose and glycine.

In one embodiment, the tonicity agent is glycerin.

In one embodiment, the composition comprises a concentration in osmotic agent between 20 and 500 mM.

In one embodiment, the concentration of tonicity agent is between 30 and 400 mM.

In one embodiment, the concentration of tonicity agent is between 50 and 250 mM.

In one embodiment, the composition according to the invention contains from 3.5 mg/mL to 10.5 mg/mL of A21G human insulin, 50 mM of phenol, 50 to 250 mM of glycerin at a pH of 7.4. This composition may further contain from 200 to 900 μM of zinc.

In one embodiment, the composition according to the invention contains 3.5 mg/mL of A21G human insulin, 50 mM of phenol, 150 to 250 mM of glycerin, 10 mM of Tris buffer at a pH of 7.4. This composition may also contain 200 to 250 μM of zinc. This composition may further comprise polysorbate 20, in particular at 8 μM or poloxamer 188, in particular at a concentration of 1.2 mg/m L.

In one embodiment, the composition according to the invention contains 3.5 mg/mL of A21G human insulin, 50 mM of phenol, 50 to 100 mM of glycerin, 50 mM of Tris buffer at a pH of 7.4. This composition may further contain 200 to 250 μM zinc. This composition may further comprise polysorbate 20, in particular at 8 μM or poloxamer 188, in particular at a concentration of 1.2 mg/mL.

In one embodiment, the composition according to the invention contains 7.0 mg/mL of A21G human insulin, 50 mM of phenol, 150 to 250 mM of glycerin, at a pH of 7.4. This composition may further contain 400 to 500 μM zinc. This composition may further comprise polysorbate 20, in particular at 8 μM or poloxamer 188, in particular at a concentration of 1.2 mg/mL.

In one embodiment, the composition according to the invention contains 10.5 mg/mL of A21G human insulin, 50 mM of phenol, 150 to 250 mM of glycerin, at a pH of 7.4. This composition may further contain 600 to 750 μM zinc. This composition may further comprise polysorbate 20, in particular at 8 μM or poloxamer 188, in particular at a concentration of 1.2 mg/mL.

In one embodiment, the composition according to the invention contains from 3.5 mg/mL to 10.5 mg/mL of A21G human insulin, 25 mM of m-cresol, 150 to 250 mM of glycerin at a pH of 7.4. This composition may further contain from 200 to 1000 μM zinc.

In one embodiment, the composition according to the invention contains 3.5 mg/mL of A21G human insulin, 50 mM of phenol, 150 to 250 mM of glycerin, 10 mM of Tris buffer at a pH of 7.4. This composition may further contain 200 to 250 μM zinc. This composition may further comprise polysorbate 20, in particular at 8 μM or poloxamer 188, in particular at a concentration of 1.2 mg/mL.

In one embodiment, the composition according to the invention contains 3.5 mg/mL of A21G human insulin, 25 mM of m-cresol, 150 to 250 mM of glycerin, 10 mM of Tris buffer at a pH of 7.4. This composition may further contain 200 to 250 μM zinc. This composition may further comprise polysorbate 20, in particular at 8 μM or poloxamer 188, in particular at a concentration of 1.2 mg/mL.

In one embodiment, the composition according to the invention contains 7.0 mg/mL of A21G human insulin, 25 mM of m-cresol, 150 to 250 mM of glycerin, at a pH of 7.4. This composition may further contain 400 to 500 μM zinc. This composition may further comprise polysorbate 20, in particular at 8 μM or poloxamer 188, in particular at a concentration of 1.2 mg/mL.

In one embodiment, the composition according to the invention contains 10.5 mg/mL of A21G human insulin, 25 mM of m-cresol, 150 to 250 mM of glycerin, at a pH of 7.4. This composition may further contain 600 to 750 μM zinc. This composition may further comprise polysorbate 20, in particular at 8 μM or poloxamer 188, in particular at a concentration of 1.2 mg/mL.

According to one embodiment, the compositions described above comprise polysorbate 20, in particular at 8 μM.

In one embodiment, the invention relates to a composition in the form of an injectable aqueous solution comprising at least A21G human insulin and a GLP-1 receptor agonist or a GLP-1 analog.

In one embodiment, GLP-1, GLP-1 analogs, or GLP-1 RA are chosen from the group consisting of exenatide (Byetta®, ASTRA-ZENECA), lixisenatide (Lyxumia®, SANOFI), their analogs or derivatives and their pharmaceutically acceptable salts.

The exenatide and lixisenatide, respectively disclosed in applications US2004/0023871 and WO0104156, are generally considered to be GLP-1 receptor agonists.

In one embodiment, the GLP-1, GLP-1 analog, or GLP-1 RA is exenatide or Byetta®, its analogs or derivatives and their pharmaceutically acceptable salts.

In one embodiment, the GLP-1, GLP-1 analog, or GLP-1 RA is lixisenatide or Lyxumia®, its analogs or derivatives and their pharmaceutically acceptable salts.

In one embodiment, the invention relates to a composition in the form of an injectable aqueous solution, the pH of which is between 7.2 and 8.0, comprising at least A21G human insulin and a GLP-1 receptor agonist or a GLP-1 analog.

According to one embodiment, said GLP-1 receptor agonist is exenatide.

According to another embodiment, said GLP-1 receptor agonist is lixisenatide.

In one embodiment, the invention relates to a composition in the form of an injectable aqueous solution, the pH of which is from 7.2 to 7.6, comprising at least A21G human insulin and a GLP-1 receptor agonist or a GLP-1 analog.

According to one embodiment, said GLP-1 receptor agonist is exenatide.

According to another embodiment, said GLP-1 receptor agonist is lixisenatide.

In one embodiment, the invention relates to a composition in the form of an injectable aqueous solution, the pH of which is 7.4, comprising at least A21G human insulin and a GLP-1 receptor agonist or a GLP-1 analog.

According to one embodiment, said GLP-1 receptor agonist is exenatide.

According to another embodiment, said GLP-1 receptor agonist is lixisenatide.

In one embodiment, the composition further comprises a nicotinic compound or a derivative thereof.

In one embodiment, the composition comprises nicotinamide.

In one embodiment, the concentration of nicotinamide is between 10 and 160 mM.

In one embodiment, the concentration of nicotinamide is between 20 and 150 mM.

In one embodiment, the concentration of nicotinamide is between 40 and 120 mM.

In one embodiment, the concentration of nicotinamide is between 60 and 100 mM.

In one embodiment, the pharmaceutical composition further comprises at least one absorption promoter chosen from absorption promoters, diffusion promoters or vasodilating agents, alone or as a mixture.

Absorption promoters include, but are not limited to, surfactants, for example, bile salts, fatty acid salts or phospholipids; nicotinic agents, such as nicotinamides, nicotinic acids, niacin, niacinamide, vitamin B3 and their salts; pancreatic trypsin inhibitors; magnesium salts; polyunsaturated fatty acids; didecanoyl phosphatidylcholine; aminopolycarboxylates; tolmetin; sodium caprate; salicylic acid; oleic acid; linoleic acid; eicosapentaenoic acid (EPA); docosahexaenoic acid (DHA); benzylic acid; nitrogen monoxide donors, for example 3-(2-hydroxy-1-(1-methylethyl)-2-nitrosohydrazino)-1-propanamine, N-ethyl-2-(1-ethyl-hydroxy-2-1-nitrosohydrazino)-ethanamine, or S-nitroso-N-acetylpenicillamine; bile acids, glycine in its conjugated form with a bile acid; sodium ascorbate, potassium ascorbate; sodium salicylate, potassium salicylate, acetyl-salicylic acid, salicylosalicylic acid, aluminum acetylsalicylate, choline salicylate, salicylamide, lysine acetylsalicylate; exalamide; diflunisal; ethenzamide; EDTA; alone or in combination.

In one embodiment, the pharmaceutical composition further comprises at least one diffusion promoter. Examples of diffusion promoters include, but are not limited to, glycosaminoglycanases, such as hyaluronidase.

In one embodiment, the pharmaceutical composition further comprises at least one vasodilator.

In one embodiment, the pharmaceutical composition further comprises at least one vasodilator causing hyperpolarization by blocking calcium ion channels.

In one embodiment, the vasodilator causing hyperpolarization by blocking calcium ion channels is adenosine, an endothelium-derived hyperpolarizing agent, a phosphodiesterase type 5 (PDE5) inhibitor, a potassium channel opening agent or any combination thereof.

In one embodiment, the pharmaceutical composition further comprises at least one cAMP-mediated vasodilating agent.

In one embodiment, the pharmaceutical composition further comprises at least one cGMP-mediated vasodilating agent.

In one embodiment, the pharmaceutical composition further comprises at least one vasodilator selected from the group comprising vasodilators which act by causing hyperpolarization by blocking calcium ion channels, cAMP-mediated vasodilators, and cGMP-mediated vasodilators.

The at least one vasodilator is selected from the group comprising nitrogen monoxide donors, for example, nitroglycerin, isosorbide dinitrate, isosorbide mononitrate, amyl nitrate, erythrityl, tetranitrate, and nitroprusside; prostacyclin and its analogs, for example epoprostenol sodium, iloprost, epoprostenol, treprostinil or selexipag; histamine, 2-methylhistamine, 4-methylhistamine; 2-(2-pyridyl)ethylamine, 2-(2-thiazolyl)ethylamine; papaverine, papaverine hydrochloride; minoxidil; dipyridamole; hydralazine; adenosine, adenosine triphosphate; uridine trisphosphate; GPLC; L-carnitine; arginine; prostaglandin D2; potassium salts; and in some cases the α1 and α2 receptor antagonists, such as prazosin, phenoxybenzamine, phentolamine, dibenamine, moxisylyte hydrochloride and tolazoline), betazole, dimaprit; receptor agonists (β2, e.g., isoproterenol, dobutamine, albuterol, terbutaline, aminophylline, theophylline, caffeine; alprostadil, ambrisentan; cabergoline; diazoxide; dihydralazine mesylate; diltiazem hydrochloride; enoximone; flunarizine hydrochloride; Ginkgo biloba extract; levosimendan; molsidomine; naftidrofuryl acid oxalate; nicorandil; pentoxifylline; phenoxyibedenzamine chloride; base piribedil; piribedil mesylate; regadenoson monohydrate; riociguat; sildenafil citrate, tadalafil, vardenafil hydrochloride trihydrate; trimetazidine hydrochloride; trinitrin; verapamil hydrochloride; endothelin receptor antagonists, for example avanafil and bosentran monohydrate; and calcium channel blockers, for example, amLodipine, aranidipine, azelnidipine, barnidipine, benidipine, cilnidipine, clevidipine, isradipine, efonidipine, felodipine, lacidipine, lercanidipine, manidipine, nicardipine, nifedipine, nilvadipine, nimodipine, nisoldipine, nitrendipine, prandipine; alone or in combination.

The compositions according to the invention may also comprise all the excipients in accordance with the Pharmacopoeias, in particular EP and/or US, and compatible with the insulins used at the usual concentrations.

According to one embodiment, the composition may be in solid or lyophilized form. This composition may then be used to reconstitute a solution or a formulation.

The modes of administration considered are by the intravenous, subcutaneous, intradermal or intramuscular route.

According to a particular embodiment, the mode of administration is the subcutaneous route.

The transdermal, oral, nasal, vaginal, ocular, buccal, pulmonary administration routes are also considered.

The invention also relates to a pump, which can be implanted or transported, comprising a composition according to the invention.

The invention also relates to the use of a composition according to the invention intended to be placed in an implantable or transportable pump.

In one embodiment, the pump is an implantable pump under the skin. Among these pumps, mention may be made of pumps allowing administration of insulin by the intraperitoneal route.

In one embodiment, the pump delivering the composition intraperitoneally according to the invention is the MiniMed Medtronic MIP2007 pump.

The invention also relates to single-dose formulations.

In one embodiment, the formulations are in the form of an injectable solution.

The preparation of a composition according to the invention has the advantage of being able to be carried out by simple mixing of A21G human insulin in an aqueous solution or lyophilized form.

The composition of the mixture is adjusted in excipients such as glycerin, m-cresol and/or phenol, zinc chloride, and polysorbate 20 (Tween® 20). This addition may be carried out by adding concentrated solutions of said excipients.

In one embodiment, the compositions are characterized in that said compositions are clear at a pH of 7.4 and have physical and chemical stability of at least one month at 40° C.

In one embodiment, the compositions are characterized in that said compositions have a physical and chemical stability greater than that of a commercial prandial insulin.

Insulin and insulin analogs can be obtained by methods of recombinant DNA technologies in bacteria such as Escherichia Coli or in yeasts such as Saccharomyces Cerevisiae (see, for example, G. Walsh Appl. Microbiol. Biotechnol. 2005, 67, 151-159). Usually, proinsulin is produced which is then digested by enzymes such as trypsin and carboxypetidase B to obtain the desired sequence.

To manufacture of A21G human insulin, the proinsulin is coded to have the glycine at A21 and, after digestion with trypsin and carboxypeptidase B, the desired insulin is obtained. A modus operandi is described by Kohn et al., In Peptides 2007, 28, 935-948.

In one embodiment, the composition according to the invention is characterized in that the A21G insulin is obtained from A21G human insulin, B31R, B32R (insulin glargine).

In one embodiment, the composition according to the invention is characterized in that the A21G insulin is obtained from A21G human insulin, B31R, B32R (insulin glargine) reacted with rat carboxypeptidase B at an insulin/carboxypeptidase ratio of between 500 and 2000, at a pH of 7.5 to 8.5 and at a temperature of 20 to 30° C. for 10 to 20 hours. The product can then be purified. This purification can be carried out by liquid chromatography.

Therefore, a route used for preparing A21G human insulin can be removing the two arginines from insulin glargine by digestion with a carboxypeptidase B. After enzymatic digestion, A21G human insulin can be purified by chromatography then isolated by lyophilization or by crystallization by conventional methods.

### DESCRIPTION OF FIGURES

FIG. 1 represents the pharmacodynamics of composition B5-1 at t0 (i.e., stored at 4° C.) and at t12 weeks (composition B5-1 “aged” at 40° C. for 12 weeks) and composition B4, commercial formulation of human insulin Humulin®.

In this figure the x-axis represents the time after injection (in minutes) and the y-axis represents blood glycemia (in mg/dL).

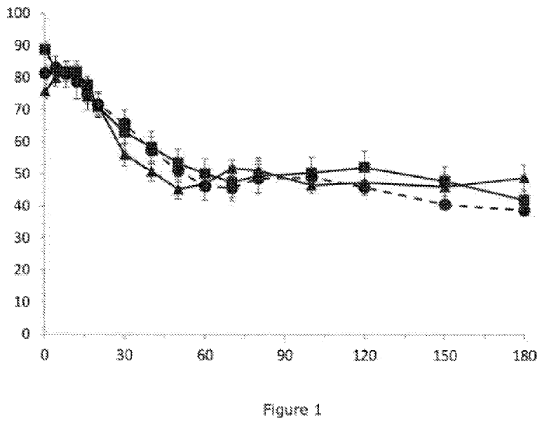

### FIG. 1:

Are shown in FIG. 1 the pharmacodynamic results obtained with composition B5-1 administered at t0 (curve plotted with squares) and at t12 weeks at 40° C. (curve plotted with triangles) in comparison with the results obtained with composition B4 (reference human insulin) (curve plotted in dotted lines with circles).

The following examples illustrate the invention in a non-limiting manner.

### EXAMPLES

### A Chemistry

### Example A1: Preparation of A21G Human Insulin (A21G Insulin)

Insulin glargine (5 g; Gan & Lee Pharmaceuticals) was mixed with the enzyme carboxypeptidase B (Reference 08039852001; Sigma-Aldrich) at a 1:500 ratio (w/w) at pH 8.0 (pH adjusted by adding trishydroxymethylaminomethane buffer), the final insulin glargine concentration being about 4 mg/m L.

The solution was left under gentle stirring at 25° C. for 17 h. The mixture was then purified by liquid chromatography, dialyzed against 0.01 N hydrochloric acid then lyophilized.

A21G human insulin was obtained with a purity of 98% and an approximately 90% yield of.

The molar mass of A21G insulin measured by mass spectrometry (Maldi-Tof) is 5752 Da.

### B Compositions

### Example B2: Composition of Rapid Insulin Analog (Humalog®) at 100 U/mL

This composition is a commercial solution of insulin lispro marketed by ELI LILLY under the name Humalog®. This product is a rapid insulin analog. The excipients in Humalog® are metacresol (3.15 mg/mL), glycerol (16 mg/mL), disodium phosphate (1.88 mg/mL), zinc oxide (to obtain 0.0197 mg zinc ion/mL), sodium hydroxide and hydrochloric acid for pH adjustment (pH 7-7.8) and water.

### Example B3: Composition of Slow Insulin Analog (Lantus®) at 100 U/mL

This composition is a commercial solution of insulin glargine marketed by SANOFI under the name Lantus®. This product is a slow insulin analog. The excipients in Lantus® are zinc chloride (30 μg/mL), m-cresol (2.7 mg/mL), glycerol (20 mg/mL), polysorbate 20 (16 μM), hydroxide sodium and hydrochloric acid for pH adjustment (pH 4) and water.

### Example B4: Composition of Human Insulin (Humulin® R) at 100 IU/mL

This composition is a commercial human insulin solution from ELI LILLY sold under the name Humulin® R. This product is human insulin. The excipients of Humulin® R are glycerol, metacresol, sodium hydroxide and hydrochloric acid for pH adjustment (pH 7.0-7.8) and water.

### Example B5: Preparation of Compositions of A21G Insulin at Concentrations of Between 100 and 300 U/mL and Excipients

A21G human insulin compositions were prepared according to the following protocol.

An aqueous solution of A21G human insulin at pH 7.3±0.2 is prepared from the lyophilizate obtained in Example A1 and an amount of sodium hydroxide or hydrochloric acid necessary for pH adjustment. This solution is added to a mixture of concentrated solutions of the excipients (zinc chloride, preservative, osmotic agent, trishydroxymethylaminomethane, arginine). A surfactant solution is then added to the mixture. The pH is measured and adjusted, if necessary, between 7.1±0.1 and 8.0±0.1 by adding concentrated solutions of sodium hydroxide or hydrochloric acid. A clear solution is obtained which does not contain visible particles. This inspection is carried out according to the recommendations of the European Pharmacopoeia (EP 2.9.20) and the U.S. Pharmacopoeia (USP <790>). The preservative is either phenol (25 to 50 mM) or meta-cresol (25 mM). The concentrations of the stock solutions are chosen so as to obtain a concentration of trishydroxymethylaminomethane from 0 to 50 mM and/or arginine from 0 to 50 mM in the final composition. The concentration of osmotic agent stock solution, here glycerol, is chosen so as to obtain a glycerol concentration of between 50 and 210 mM in the final composition. The surfactant used may be Tween® 20, at a concentration of at least 8 μM, or Poloxamer® 188, at a concentration of 1.2 mg/mL, in the final composition. The concentration of A21G human insulin, C_{IHA21G}, is between 100 and 300 U/mL. The zinc chloride concentration (μM) corresponds to 2 to 3 times the concentration of A21G human insulin (U/m L), i.e., C_{ZnCl2} (μM)=2-3 C_{IHA21G} (U/mL).

These compositions are presented in Table 1 below.

### Example B6: Preparation of Compositions of A21G Human Insulin at Concentrations of Between 100 and 300 U/mL (Corresponding to 3.5 and 10.5 mg/mL) and of Excipients at pH 4

These compositions were produced according to the protocol described in Example B5 from an aqueous solution of A21G human insulin at pH 4.0±0.2, prepared from the lyophilizate obtained in Example A1 and an amount of sodium hydroxide or hydrochloric acid allowing adjustment of the pH. The concentration of trishydroxymethylaminomethane is between 0 and 50 mM in the final composition. The concentrations of the other excipients are similar to those mentioned in Example B5.

These compositions are presented in Table 1 below.

TABLE 1
A21G insulin compositions prepared according to protocols
B5 (B5-1 to B5-13) and B6 (B6-1 to B6-3)

| Com- | | | | | | Gly- | |
|---|---|---|---|---|---|---|---|
| po- | Insulin | Tris | | | [Zn] | cerol | pH |
| sition | (mg/mL | (mM) | Additive | Surfactant | (μM) | (mM) | (±0.1) |
| B5-1 | 3.5 | 10 | Phenol | Tween ® 20 | 230 | 200 | 7.4 |
| | | | (50 mM) | | | | |
| B5-2 | 3.5 | 10 | Phenol | Poloxamer ® | 230 | 184 | 7.4 |
| | | | (50 mM) | 188 | | | |
| | | | | (1.2 mg/mL) | | | |
| B5-3 | 3.5 | — | m-cresol | Poloxamer ® | 300 | 184 | 7.4 |
| | | | (25 mM) | 188 | | | |
| | | | | (1.2 mg/mL) | | | |
| B5-4 | 3.5 | 10 | Phenol | Tween ® 20 | 230 | 200 | 7.0 |
| | | | (50 mM) | (8 μM) | | | |
| B5-5 | 3.5 | 10 | Phenol | Tween ® 20 | 230 | 200 | 7.2 |
| | | | (50 mM) | (8 μM) | | | |
| B5-6 | 3.5 | 10 | Phenol | Tween ® 20 | 230 | 200 | 7.5 |
| | | | (50 mM) | (8 μM) | | | |
| B5-7 | 3.5 | 10 | Phenol | Tween ® 20 | 230 | 200 | 8.0 |
| | | | (50 mM) | (8 μM) | | | |
| B5-8 | 10.5 | — | m-cresol | Poloxamer ® | 900 | 184 | 7.4 |
| | | | (25 mM) | 188 | | | |
| | | | | (1.2 mg/mL) | | | |
| B5-9 | 3.5 | — | Phenol | Tween ® 20 | 230 | 184 | 7.4 |
| | | | (50 mM) | (8 μM) | | | |
| | | | Arginine | | | | |
| | | | (50 mM) | | | | |
| B5-10 | 7 | 10 | Phenol | Tween ® 20 | 460 | 184 | 7.4 |
| | | | (50 mM) | (8 μM) | | | |
| B5-11 | 10.5 | 10 | Phenol | Tween ® 20 | 690 | 184 | 7.4 |
| | | | (50 mM) | (8 μM) | | | |
| B5-12 | 3.5 | 50 | Phenol | Tween ® 20 | 230 | 60 | 7.4 |
| | | | (50 mM) | (8 μM) | | | |
| | | | Arginine | | | | |
| | | | (40 mM) | | | | |
| B6-1 | 3.5 | — | m-cresol | Poloxamer ® | 300 | 184 | 4.0 |
| | | | (25 mM) | 188 | | | |
| | | | | (1.2 mg/mL) | | | |
| B6-2 | 3.5 | — | m-cresol | Poloxamer ® | — | 184 | 4.0 |
| | | | (25 mM) | 188 | | | |
| | | | | (1.2 mg/mL) | | | |
| B6-3 | 10.5 | — | m-cresol | Tween ® 20 | — | 184 | 4.0 |
| | | | (25 mM) | (8 μM) | | | |
| B5-13 | 10.5 | 10 | Phenol | Tween ® 20 | 690 | 200 | 7.4 |
| | | | (50 mM) | (8 μM) | | | |

### C Stability

### Example C1: Study of Physical Stability at 50° C. of the Compositions Prepared in Comparison with Commercial Insulins at a Concentration of 100 U/mL in Vials

At least five 3-mL vials filled with 1 mL of composition are placed vertically in an oven maintained at 50° C. The vials are visually inspected at least weekly for the appearance of visible particles or turbidity. This inspection is carried out according to the recommendations of the European Pharmacopoeia (EP 2.9.20) and the U.S. Pharmacopoeia (USP <790>): the vials are subjected to lighting of at least 2000 Lux and are observed against a white background and a black background. The number of weeks of stability corresponds to the time during which at least half of the vials remain clear and contain no visible particles on inspection.

The physical stability results obtained with different compositions are presented in the table below.

TABLE 2
Results of physical stability studies at 50° C. of compositions
B5-1 to B5-3, B5-9, B5-12, B6-1 to B6-3 and B2 to B4.

| | | Insulin | | |
|---|---|---|---|---|
| | | concentration | | |
| Composition | Insulin | (mg/mL) | pH | 50° C. |
| B5-1 | A21G | 3.5 | 7.4 | No particles at 15 |
| | | | | weeks |
| B5-2 | A21G | 3.5 | 7.4 | No particles at 15 |
| | | | | weeks |
| B5-3 | A21G | 3.5 | 7.4 | No particles at 8 |
| | | | | weeks |
| B5-9 | A21G | 3.5 | 7.4 | No particles at 9 |
| | | | | weeks |
| B5-12 | A21G | 3.5 | 7.4 | No particles at 6 |
| | | | | weeks |
| B6-1 | A21G | 3.5 | 4.0 | Particles at 1 |
| | | | | week |
| B6-2 | A21G | 3.5 | 4.0 | Particles at 1 |
| | | | | week |
| B6-3 | A21G | 3.5 | 4.0 | Particles at 4 |
| | | | | weeks |
| B2 (Humalog ®) | Insulin lispro | 3.5 | 7.4 | Particles at 1 |
| | | | | week |
| B3 (Lantus ®) | Insulin glargine | 3.6 | 4.0 | Particles at 1 |
| | | | | week |
| B4 | Human insulin | 3.5 | 7.4 | Particles at 2 |
| (Humulin ® R) | | | | weeks |

The human insulin compositions A21G at pH 4.0 (B6-1 to B6-3) and the commercial formulations (B2 to B4) are less stable at 50° C. than compositions containing A21G human insulin at pH 7.4 (B5-1 to B5-3, B5-9, and B5-12). These compositions (B5-1 to B5-3, B5-9, and B5-12) make it possible to obtain a physical stability of at least 6 weeks at 50° C.

### Example C2: Chemical Stability Study at 40° C. and 50° C.

The compositions, presented in the following table, are placed in an oven maintained at 40° C. or 50° C.

The compositions are analyzed at 4, 8, 12 weeks at 40° C. and at 2 weeks at 50° C. At the end of each time interval, a sample is analyzed by RP-HLPC-UV (214 nm) with a Cis column and a phosphate/acetonitrile buffer elution gradient in the presence of sodium heptanesulphonate to determine by surface percentage the proportion of deamidated products dB3/disoB3 as well as other impurities (designated total impurities). At the end of each time interval, a sample is also analyzed by SE-HPLC-UV (276 nm) with a silica column coated with 80-A pores and a water/acetonitrile/trifluoroacetic acid mobile phase to determine by surface percentage the proportion of high molecular weight products (HMWP).

The results are given as a percentage in the table below.

TABLE 3
Results of the chemical stabilities of solutions
B5-1 to B5-3, B5-9, and B5-12 and commercial
human insulin compositions Humulin ® R

| Com- | | | | | | | |
|---|---|---|---|---|---|---|---|
| po- | Insulin | Degradation | | 40° C. | 40° C. | 40° C. | 50° C. |
| sition | (mg/mL) | products | T0 | 4 W | 8 W | 12 W | 2 W |
| B5-1 | A21G | dB3/disoB3 | 0.4 | 1.4 | 2.8 | 4.2 | 1.8 |
| | (3.5) | HMWP | 0.1 | 0.3 | 0.7 | 0.9 | 0.3 |
| | | Total imp.* | 0.3 | 1.9 | 3.7 | 5.0 | 2.2 |
| B5-2 | A21G | dB3/disoB3 | 0.1 | — | — | — | 2.0 |
| | (3.5) | HMWP | 0.2 | | | | 0.5 |
| | | Total imp.* | 0.3 | | | | 2.5 |
| B5-3 | A21G | dB3/disoB3 | 0.2 | 3.0 | 5.7 | 8.7 | 4.2 |
| | (3.5) | HMWP | 0.1 | 0.4 | 1.0 | 1.9 | 0.5 |
| | | Total imp.* | 0.6 | 1.4 | 4.1 | 5.2 | 2.3 |
| B5-9 | A21G | dB3/disoB3 | 0.1 | — | — | — | 1.8 |
| | (3.5) | HMWP | 0.1 | | | | 0.1 |
| | | Total imp.* | 0.3 | | | | 2.5 |
| B5-12 | A21G | dB3/disoB3 | 0.1 | 1.4 | 2.8 | 4.1 | 1.6 |
| | (3.5) | HMWP | 0.1 | 0.2 | 0.2 | 0.3 | 0.1 |
| | | Total imp.* | 0.4 | 2.3 | 4.3 | 5.6 | 2.5 |
| B4 | Human | dB3/disoB3 | 2.5 | 11.7 | — | — | 6.4 |
| | insulin | HMWP | 0.3 | 2.1 | — | — | 3.4 |
| | (3.5) | Total imp.* | 1.4 | 12.8 | — | — | 6.5 |

*Total imp. means all impurities except dB3/disoB3 and HMWP
“—” means not measured

The commercial composition Humulin® R (B4) is analyzed after 4, 8 and 12 weeks at 40° C. and after 2 weeks at 50° C. At these time intervals, this composition no longer meets the specifications of the European Pharmacopoeia in terms of physical stability (see above), nor in terms of percentage of impurities. Formulations B5-1 to B5-3, B5-9 and B5-12 are much more stable.

### Example C3: Effect of pH on the Physical and Chemical Stability of Compositions Comprising A21G Human Insulin

Formulations B5-4 to B5-7 have a composition identical to that of formulation B5-1, except for the pH.

TABLE 4
Results of the effect of pH on the physical and chemical
stability of solutions B5-4 to B5-7

| | | | Degradation | |
|---|---|---|---|---|
| | | | products after 4 weeks | |
| Composition | pH | Vial 40° C. | at 40° C. (relative to t0) | |
| B5-4 | 7.0 ± | Particles observed | — | |
| | 0.1 | immediately at t0 | | |
| B5-5 | 7.2 ± | No particles at 11 | dB3/disoB3 | 1.3% |
| | 0.1 | weeks | HMWP | 0.1% |
| | | | Total imp.* | 1.8% |
| B5-6 | 7.5 ± | No particles at 11 | dB3/disoB3 | 1.4% |
| | 0.1 | weeks | HMWP | 0.2% |
| | | | Total imp.* | 1.9% |
| B5-7 | 8.0 ± | No particles at 11 | dB3/disoB3 | 1.6% |
| | 0.1 | weeks | HMWP | 0.3% |
| | | | Total imp.* | 2.4% |

*“total impurities” means all the impurities except dB3/disoB3 and HMWP
—means not measured

The compositions have good stability between 7.2 and 8.0. It should be noted that even better stability at 40° C. is obtained in a pH range of between 7.2 and 7.5.

### Example C4: Physical and Chemical Stability of A21G Human Insulin at 100 and 300 U/mL (i.e., 3.5 and 10.5 mg/mL), at pH 7.4

TABLE 5
Results of the physical stability at 50° C. of the compositions at
100 and 300 U/mL of A21G insulin (B5-3 and B5-8).

| | A21G insulin | |
|---|---|---|
| Formulation | (mg/mL) | 50° C. |
| B5-3 | 3.5 | No particles at 8 weeks |
| B5-8 | 10.5 | No particles at 4 weeks |
| | | Particles at 5 weeks |

TABLE 6
Results of the chemical stabilities at 40 and
50° C. of the compositions at 100 and
300 U/mL of A21G insulin (B5-3 and B5-8).

| Com- | A21G | Deg- | | | | | |
|---|---|---|---|---|---|---|---|
| po- | insulin | radation | | 40° C. | 40° C. | 40° C. | 50° C. |
| sition | (mg/mL) | products | T0 | 4 W | 8 W | 12 W | 2 W |
| B5-3 | 3.5 | dB3/disoB3 | 0.2 | 3.0 | 5.7 | 8.9 | 4.2 |
| | | HMWP | 0.1 | 0.4 | 1.0 | 1.9 | 2.3 |
| | | Total imp. | 0.6 | 1.4 | 4.1 | 5.2 | 0.5 |
| B5-8 | 10.5 | dB3/disoB3 | 0.4 | 1.9 | 3.8 | 5.9 | 2.7 |
| | | HMWP | 0.1 | 0.2 | 0.5 | 0.9 | 2.0 |
| | | Total imp. | 1.2 | 1.3 | 3.1 | 3.9 | 0.4 |

“*” “Total imp.” means all impurities except dB3/disoB3 and HMWP

The results are expressed as a percentage in the table below.

The results presented above show that composition B5-8 (U300) has good physical stability and excellent chemical stability.

### Example C5: Physical and Chemical Stability of A21G Human Insulin at 100 and 300 U/mL (i.e., 3.5 and 10.5 mg/mL) in Cartridges (3 mL), at 40° C.

The compositions B5-1 and B5-13 of A21G human insulin are inserted into 3-mL cartridges and placed in an oven at 40° C. under static conditions. Cartridges are visually inspected every two weeks for the appearance of visible particles or turbidity. This inspection is carried out according to the recommendations of the European Pharmacopoeia (EP 2.9.20) and the U.S. Pharmacopoeia (USP <790>) described for solutions in vials. Thus, the cartridges are subjected to lighting of at least 2000 Lux and are observed against a white background and a black background. The number of weeks of stability corresponds to the time during which at least half of the cartridges remain clear and do not contain visible particles on inspection.

The cartridges are analyzed at 4, 8 and 12 weeks at 40° C. At the end of each time interval, a cartridge is taken and an aliquot is analyzed by RP-HLPC-UV (214 nm) with a C18 column and a phosphate/acetonitrile buffer elution gradient in the presence of sodium heptanesulphonate to determine by surface percentage the proportion of deamidated products dB3/disoB3 as well as other impurities (designated total impurities). At the end of each time interval, an aliquot is also analyzed by SE-HPLC-UV (276 nm) with a silica column coated with 80-A pores and a mobile phase of water/acetonitrile/trifluoroacetic acid to determine by surface percentage the proportion of high molecular weight products (HMWP).

The results are given as a percentage in the table below.

TABLE 6a
Results of the physical stability at 40°C. of the compositions at
100 and 300 U/mL of A21G insulin (B5-1 and B5-13).

| | A21G insulin | |
|---|---|---|
| Composition | (mg/mL) | 40° C. |
| B5-1 | 3.5 | No particles at 12 weeks |
| B5-13 | 10.5 | No particles at 10 weeks |

TABLE 6b
Results of the chemical stabilities of solutions B5-1 and B5-13.

| Com- | | Deg- | | | | | |
|---|---|---|---|---|---|---|---|
| po- | Insulin | radation | | 40° C. | 40° C. | 40° C. | 40° C. |
| sition | (mg/mL) | products | T0 | 4 W | 8 W | 11 W | 12 W |
| B5-1 | A21G | dB3/disoB3 | 0.1 | 1.4 | 2.6 | — | 3.8 |
| | (3.5) | HMWP | 0.2 | 0.3 | 0.4 | — | 0.7 |
| | | Total imp.* | 0.6 | 1.9 | 3.1 | — | 4.5 |
| B5-13 | A21G | dB3/disoB3 | 0.3 | — | — | 3.2 | — |
| | (10.5) | HMWP | 0.2 | — | — | 0.5 | — |
| | | Total imp.* | 0.4 | — | — | 4.0 | — |

*Total imp. means all the impurities except dB3/disoB3 and HMWP

The results are expressed as a percentage in the table below.

The results presented above show that the compositions B5-1 (U100) and B5-13 (U300) have excellent physical and chemical stability.

### D Pharmacodynamics

### Example D1: Protocol for Measuring the Pharmacodynamics of Insulin Solutions

Domestic pigs of around 50 kg, previously catheterized at the jugular, are fasted 2.5 hours before the start of the experiment. In the hour before the insulin injection, 3 blood samples are taken to determine the basal glucose level.

The injection of the insulin M1 or human insulin formulations at a dose of 0.2 μl/kg is carried out subcutaneously in the flank of the animal using an insulin pen (Novo, Sanofi or Lilly) equipped with a 31 G needle.

Blood samples are then taken at the following times: 4, 8, 12, 16, 20, 30, 40, 50, 60, 70, 80, 100, 120, 150 and 180 minutes. After each sample, the catheter is rinsed with a dilute heparin solution. A drop of blood is taken to determine the blood glucose level using a glucometer. The average blood glucose curves, expressed in mg/dL, are then plotted.

### Example D2: Pharmacodynamic Results of the Insulin Solution A21G B5-1 at Time Zero (t0, i.e., Stored at 4° C.) and after 12 Weeks at 40° C. and of a Solution of Human Insulin

TABLE 7
Compositions used for pharmacodynamics

| | A21G human insulin | Human insulin | |
|---|---|---|---|
| Composition | (U/mL) | (U/mL) | Number of pigs |
| B5-1 | 100 | — | 12 |
| B4 | — | 100 | 11 |

The pharmacodynamic results obtained with the composition B5-1 are illustrated in FIG. 1. Analysis of these profiles indicates that similar hypoglycemic actions are obtained with composition B5-1 administered at t0 (curve plotted with squares) and t12 weeks at 40° C. (curve plotted with triangles). On the other hand, composition B5-1, administered at t0 or at t12 weeks at 40° C., has a hypoglycemic action similar to that obtained with composition B4 (reference human insulin) (curve plotted in dotted lines with circles).

1. A composition in the form of an injectable aqueous solution, the pH of which is between 7.2 and 8.0 (7.2≤pH≤8.0) comprising at least A21G human insulin.

2. The composition according to claim 1, wherein the concentration of A21G human insulin is between 40 and 1000 U/mL (40 U/mL ≤concentration of A21G human insulin≤1000 U/mL).

3. The composition according to claim 1, wherein the concentration of A21G human insulin is 100 U/mL.

4. The composition according to any one of claims 1 to 3, claim 1, wherein the concentration of A21G human insulin is 300 U/mL.

5. The composition according to claim 1, wherein the zinc salt concentration is between 50 and 600 μM per 100 U/mL of A21G insulin.

6. The composition according to claim 1, wherein the zinc salt concentration is 230 μM per 100 U/mL of A21G insulin.

7. The composition according to claim 1, wherein the composition further comprises a phenolic preservative.

8. The composition according to claim 7, wherein the concentration of phenolic preservative is between 15 and 100 mM.

9. The composition according to claim 7, wherein the phenolic preservative is phenol.

10. The composition according to claim 7, wherein the phenol concentration is between 30 and 75 mM (30 mM≤phenol concentration≤75 mM).

11. The composition according to claim 10, wherein the phenol concentration is 50 mM.

12. The composition according to claim 1, wherein it further comprises a surfactant.

13. The composition according to claim 12, wherein the surfactant is chosen from polysorbates.

14. The composition according to claim 12, wherein the surfactant is chosen from polysorbate 20 or “Tween® 20”.

15. The composition according to claim 13 or 14, claim 13, wherein the polysorbate concentration is between 4 and 20 μM (4 μM≤polysorbate concentration≤20 μM).

16. The composition according to claim 1, wherein it further comprises arginine.

17. The composition according to claim 1, wherein it comprises trishydroxymethylaminomethane.

18. The composition according to claim 17, wherein it comprises trishydroxymethylaminomethane at a concentration between 2 and 100 mM (2 mM≤trishydroxymethylaminomethane concentration≤100 mM).

19. A method for treating diabetes, comprising administering the composition according to claim 1 as a bolus before meals.

20. A method for treating diabetes, comprising administering the composition according to claim 1 as prandial insulin.


SEQUENCE LISTING (2 sequences)

SEQ ID NO: 1 - protein, 21 aa, Homo sapiens
GIVEQCCTSICSLYQLENYCG

SEQ ID NO: 2 - protein, 30 aa, Homo sapiens
FVNQHLCGSHLVEALYLVCGERGFFYTPKT